(19) United States
(12) Patent Application Publication
(10) Pub. No.: US 2015/0297642 A1
(43) Pub. Date: Oct. 22, 2015

(54) COMPOSITIONS FOR THE RESTORATION OF A FECAL MICROBIOTA AND METHODS FOR MAKING AND USING THEM

(71) Applicant: Thomas Julius BORODY, Five Dock (AU)

(72) Inventor: Thomas Julius BORODY, Five Dock (AU)

(21) Appl. No.: 14/647,062

(22) PCT Filed: Nov. 26, 2013
(86) PCT No.: PCT/AU2013/001362

§ 371 Date: May 22, 2015

Related U.S. Application Data
(60) Provisional application No. 61/729,994, filed on Nov. 26, 2012.

(51) Int. Cl. A61K 35/38; C12N 1/20; A61K 45/06; A61K 9/19; A61K 35/76; A61K 35/74

(52) U.S. Cl. CPC A61K 35/38; A61K 35/76; A61K 35/74; A61K 45/06; A61K 9/19; C12N 1/20

(57) ABSTRACT

In alternative embodiments, the invention provides compositions and methods for treating various disorders and conditions in mammals, including chronic disorders in which there is a presence of an abnormal microbiota or an abnormal distribution of microflora in the gastrointestinal tract. In alternative embodiments, the invention provides liquid preparations or formulations derived from a human fecal material (e.g., a stool) processed, e.g., filtered and/or centrifuged, such that all bacteria, fungal spores and viruses are removed, but retaining the native biologically active molecules from the fecal material and bacteriophages. In alternative embodiments, the invention provides a “rough-”, “incomplete-” or medium-filtered microbiota which still comprises native physiological components or nutritive agents for the bacteria, e.g., retains native biologically and nutritionally active components. In alternative embodiments, the invention provides a highly filtered or substantially purified microbiota in combination with, or having added back, a liquid preparation or formulation of the invention. In alternative embodiments, the invention provides compositions or formulations where the bacteria, or microbiota, component has been cultured, or cultured under anaerobic conditions, or harvested, stored and/or cultured under anaerobic conditions. In alternative embodiments, the invention provides various additives, compositions and donor restrictions for treating these disorders and conditions.

### TECHNICAL FIELD

This invention generally relates to medicine and gastroenterology, pharmacology and microbiology. In alternative embodiments, the invention provides compositions and methods for treating various disorders and conditions in mammals, including chronic disorders in which there is a presence of an abnormal microbiota or an abnormal distribution of microflora in the gastrointestinal tract. In alternative embodiments, the invention provides liquid preparations or formulations derived from a human fecal material (e.g., a stool) processed, e.g., filtered and/or centrifuged, such that all bacteria, fungal spores and viruses are removed, but retaining the native biologically active molecules from the fecal material. In alternative embodiments, the invention provides a “rough-”, “incomplete-” or medium-filtered microbiota which still comprises native physiological components or nutritive agents for the bacteria; e.g., retains native biologically and nutritionally active components. In alternative embodiments, the invention provides a highly filtered or substantially purified microbiota in combination with, or having added back, a liquid preparation or formulation of the invention. In alternative embodiments, the invention provides compositions or formulations where the bacteria, or microbiota, component has been cultured, or cultured under anaerobic conditions, or harvested, stored and/or cultured under anaerobic conditions. In alternative embodiments, the invention provides various additives, compositions and donor restrictions for treating these disorders and conditions.

### BACKGROUND

The human gastrointestinal (GI) microbial flora, also called microbiota, contains around 3.3 million genes compared to around 25,000 genes the human microbiome contains. The total composition of these genes is called the Human Gut Microbiome. The wild type, or normal, GI microbiota contains in excess of 1,200 to 1,500 various species of bacteria and a small number of viruses and some fungi. There are other components in the GI microbiota, including fibre proteins, small levels of unabsorbed carbohydrates, mucus, ash, mineral salts, trace elements, fats, micronutrients, dead bacteria and at times undigested food.

The microbiota in terms of cell numbers make up a very large component of the living structures of the human body. The absolute numbers of living bacterial cells within the GI microbiota are said to be around 9 times more than all the living cells in the human body. Indeed, by cell count, we are 10% human and 90% bacterial flora.

Human GI microbiota are therefore considered a ‘virtual organ’ which has the characteristics of a body organ being ‘living’, being within our body and having characteristics of organogenesis after birth, anatomy, physiology, pathology, and other features. The GI microbiota has the potential for being maldeveloped or being infected with various parasites, viruses, fungi or bacteria. Hence, treatments for such an organ need to be developed. Apart from antibiotics and as with other organs, transplantation is one possible treatment.

It is this GI microbiota when invaded by various pathogens that can remain transiently or long term, even lifelong in the flora—that constitute the various illnesses referable to the small and large bowel which we have previously not realised are caused by ‘superinfection’ of our largest organ, for example, causing Irritable Bowel Syndrome (IBS), C. difficile infection (CDI), diarrhea, pseudomembranous colitis and others.

There has recently been an enormous expansion in our understanding of bowel flora related conditions. Some of these conditions are easily understandable to be caused by abnormal bacteria e.g. Salmonella enteritis, whereas others such as obesity are more difficult to comprehend in terms of the mechanisms that might be playing a role in the causality of obesity yet originating in the bowel flora. Nonetheless, there is a growing list of various conditions that are now becoming tied to the GI microbiota, e.g., the intestinal microbiome. It is now recognized that conditions such as IBS, Colitis, Crohn's Disease, constipation, Metabolic Syndrome to name a few, are in no small part caused by abnormal bowel flora, or abnormal GI microbiota.

There is activity in the research community to attempt to repair the flora and to try and improve or cure such conditions that might be mediated by an abnormal GI microbiota, i.e., an abnormal bacterial composition populating the bowel. Antibiotics can give transient improvement, but often fail (for example, recurrent CDI), and these failures point to a need for a fresh approach to treatment.

A common underlying factor shared by all such disorders is that their onset is after some extraneous invading GI infection, albeit the patients may not remember this as it might have occurred decades before, for example, as with IBS or constipation. In many of these conditions the infection cannot be demonstrated by culture as there are more than 3.3 million different microbial genes and commensurate large numbers of sub-species in the human GI microbiota, the GI flora, and so the diversity of microbial sub-species level composition is quite enormous, and only a small percentage of these can be cultured. It is therefore unlikely that the majority of these conditions are the result of a simple super-infection with a specific single pathogen, e.g., as with CDI, and it is also unlikely that any time soon it can be determined which specific species and/or subspecies are responsible (pathogenic) for or involved in any particular disease or condition. Hence, the current thinking is that perhaps the best available therapy (e.g., with CDI) should be simply to find a proper composition and delivery system/s for infusing donor flora so that occult or pathogenic bacterial overgrowth conditions can be reversed or ameliorated.

Fecal Microbiota Transplantation (FMT) previously known as “Fecal Bacteriotherapy” (see, e.g., Borody (2004) J. Clin. Gast. 38:475-483) represents a therapeutic method which allows the most rapid reconstitution of the normal composition of colonic microbial communities. It has been a therapy of last resort for patients with severe CDI and particularly with relapsing CDI. FMT is now becoming much more accepted medically; however, there is a need to improve on the deficiencies of FMT-based therapeutics. While there is wide availability of good donor FMT material, design of a complex yet clinically active composition that is patient-acceptable, e.g., not resemble crude, smelling stool, but rather a more acceptable pharmaceutical-like ‘biological’ compositions that will gain wider patient and physician acceptance, is needed. Need for clinically active FMT compositions that are more acceptable to a wide patient and physician group are needed in fields where patient are not desperately ill (where acceptance is quite high). Implantation (e.g., transplantation) of crude homogenised human stool has abolished CDI, and the donor flora implants for prolonged periods of time, see, e.g., Grehan (2010) J. Clin. Gastroenterol. 44(8):551-561.

### SUMMARY

In alternative embodiments, the invention provides compositions, including formulations, pharmaceutical compositions, foods, feeds, supplements, products of manufacture, and the like, comprising a treated or untreated human GI microbiota, or a partially, substantially or completely isolated human GI microbiota; and methods of making and using them.

In alternative embodiments, the invention provides the following compositions, and methods for making, storing and using them, and the invention provides methods and uses for the following compositions:

### (1) A Liquid Preparation

In alternative embodiments, the invention provides liquid preparations or formulations derived from a human fecal material (e.g., a stool) processed, e.g., filtered and/or centrifuged, such that all bacteria, fungal spores and viruses are removed, but retaining the native biologically active molecules from the fecal material, including for example, bacterial secretory products such as e.g., bacteriocins (including colicin, troudulixine or putaindicine, or microcin or subtilosin A), lanbiotics (including thuricin), nisin, subtilin, epidermin, mutacin, mersacidin, actagardine, cinnamycin), a lacticin and other pore-forming peptidic toxins; wherein these and/or other components of the liquid preparation can act as anti-spore (e.g., anti-Clostridium difficile spore), antimicrobial and/or anti-inflammatory compounds (e.g., interleukins, cytokines); and also including other biologically active molecules (BAMs) produced by bacteria or other microorganisms of the microbiota. This level of filtration will nevertheless retain bacteriophages which are of sub-viral size and will contribute to the therapeutic capacity of this filtrate.

### (2) A “Rough-”, “Incomplete-” or Medium-Filtered Microbiota

In alternative embodiments, the invention provides a “rough-”, “incomplete-” or medium-filtered microbiota which still comprises native physiological components or nutritive agents for the bacteria, e.g., retains native biologically and nutritionally active components, e.g., bacterial secretory products as discussed above, including e.g., anti-microbial (e.g., anti-spore) and anti-inflammatory products. In one embodiment, a fecal material (e.g., stool), is taken, dissolved and homogenised and passed through progressively smaller filter or a sieve sizes, stopping at to 0.1 mm sieve/filter holes; thus retaining the biologically and nutritionally active native liquid components.

This rough-”, “incomplete-” or medium-filtered microbiota exemplary embodiment is in contrast to highly purified preparations, e.g., as described by Sadowsky, et al., WO 2012/122478 A1, who prepared FMT material by filtering continued through ever smaller sieve holes until the stool was passed through a sieve down to 0.020 mm, resulting in a very highly purified microbiota mass with well over 95% of bacterial cells alone, while the surrounding native “biologically and nutritionally active” liquid material was discarded.

In alternative embodiments this exemplary “rough filtered” or “medium filtered” composition maintains its physiological and also significantly, nutritional status, by keeping its native liquid components and small fibre molecules to supply nutrients to the flora of the microbiota. In this exemplary embodiment the donor flora is left “incompletely” filtered, e.g., finally down to about 0.1 mm sieve holes, to allow for some physiological “food” to remain for the bacteria and to retain the liquid components with their anti-microbial (e.g., anti-spore) and anti-inflammatory products.

(3) Highly Filtered or Substantially Purified Microbiota with Added Back Liquid Preparations or Formulations

In alternative embodiments, the invention provides a highly filtered or substantially purified microbiota in combination with, or having added back, a liquid preparation or formulation of the invention (as summarized in category (1), above). By “adding back”, or reconstituting, to the highly filtered or substantially purified microbiota a liquid preparation or formulation of the invention, the properties of the final composition or formulation are greatly enhanced, e.g., the highly filtered or substantially purified microbiota now has the properties of a liquid preparation or formulation of the invention, e.g., as summarized in category (1).

In alternative embodiments, this embodiment uses a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material. In alternative embodiments, this embodiment uses a substantially isolated, purified, or substantially entire microbiota as described in Sadowsky, et al., WO 2012/122478 A1, or as described in Borody, et al., WO 2012/016287 A2.

(4) A Cultured and/or Anaerobic Microbiota

In alternative embodiments, the invention provides compositions or formulations where the highly filtered or substantially purified microbiota composition with the liquid component added back, as per (3) above, is placed into an enrichment culture, optionally under anaerobic conditions, or optionally harvested, stored and/or cultured under anaerobic conditions.

In alternative embodiments the bacteria or microbiota component is cultured for about 2 to about 72 hours (hrs), or about 1 hour to 24 hours, or about 30 minutes to 12 hours, to increase the numbers of the bacteria and their products without needing to use larger numbers of donors.

In alternative embodiments, the invention provides formulations or pharmaceutical preparations comprising:

(a) (i) a liquid preparation harvested from a fecal material, wherein the liquid preparation is capable of passing through an at least about 0.22 micron filter and lacks any, or substantially all, intact viruses, fungal spores and bacteria, albeit bacteriophages will remain; or

(ii) a liquid preparation made by a process comprising: (1) providing a fecal material; and (2) passing the fecal material through an at least about 0.22 micron (μ) filter such that the filtrate lacks any, or substantially all, intact viruses, fungal spores and bacteria,

wherein optionally, the fecal material is passed through a series of progressively smaller sized filters before the resulting liquid preparation is finally passed through the at least about 0.22 micron filter,

and optionally, before passing the fecal material through the at least about 0.22 micron filter, the fecal material is first centrifuged, and the supernatant is used as the liquid preparation starting material for step (i) or (ii),

and optionally, before passing the fecal material through the at least about 0.22 micron filter, and/or before centrifuging, the fecal material is first homogenized with a saline or a buffered solution,

and optionally, before passing the fecal material through the at least about 0.22 micron filter, the starting fecal material, or the after-centrifugation supernatant, is filtered with one or several filters to ultimately remove all (or substantially all) cells of bacterial origin from the liquid preparation, or to ultimately remove all cells (or substantially all) of less than about 5 micrometres (μm) diameter from the liquid preparation;

(b) the liquid preparation of (a), wherein the fecal material consists of a human fecal material;

(c) the liquid preparation of (a) or (b), further comprising by having added to the liquid preparation:, a fiber, biologically active proteins or peptides, micronutrients, fats, sugars or small carbohydrates, trace elements, mineral salts, ash, mucous, amino acids, nutrients, vitamins or minerals, or all or any combination thereof, optionally “added back” to reconstitute a “wild type” healthy flora or human microbiota environment;

(d) the liquid preparation of any of (a) to (c), further processed or formulated for either freezing or freeze-drying into a powder, or equivalent, and optionally further comprising a cryoprotectant, a lyoprotectant, or a preservative; or

(f) the liquid preparation of (d), further processed or formulated by reconstituting the frozen or freeze-dried powder in a liquid, wherein optionally the liquid is a sterile saline.

In alternative embodiments, the invention provides formulations or pharmaceutical preparations comprising:

(a) (i) a highly filtered or substantially purified microbiota, and, (ii) a liquid preparation or formulation of the invention;

(b) the formulation or pharmaceutical preparation of (a), wherein the highly filtered or substantially purified microbiota comprises or consists of a substantially isolated or a purified fecal flora or entire (or substantially entire) microbiota;

(c) the formulation or pharmaceutical preparation of (a) or (b), wherein the highly filtered or substantially purified microbiota comprises or consists of an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material;

(d) the formulation or pharmaceutical preparation of any of (a), to (c), wherein the highly filtered or substantially purified microbiota comprises or consists of a substantially isolated, purified, or substantially entire microbiota as described in Sadowsky, et al., WO 2012/122478 A1, or as described in Borody, et al., WO 2012/016287 A2;

(e) the formulation or pharmaceutical preparation of any of (a) to (d), wherein the highly filtered or substantially purified microbiota is made by using a plasmapheresis, a centrifugation, a celltrifuge, a column chromatography, an affinity chromatography, an immunoprecipitation, or antibodies fixed to a solid surface, a bead or a plate;

(f) the formulation or pharmaceutical preparation of any of (a) to (e), wherein the liquid preparation or formulation of the invention makes up between about 1% to 99%, or about 1%, 10%, 20%, 30%, 40%, 50%, 55%, 60%, 65%, 70%, 75%, 80%, 85%, 90% or 95% or more of the volume of a final formulation or pharmaceutical preparation;

(g) the liquid preparation of any of (a) to (f), further processed or formulated for either freezing or freeze-drying into a powder, or equivalent, and optionally further comprising a cryoprotectant, a lyoprotectant, or a preservative; or

(h) the liquid preparation of (g), further processed or formulated by reconstituting the frozen or freeze-dried powder in a liquid, wherein optionally the liquid is a sterile saline.

In alternative embodiments, the invention provides formulations or pharmaceutical preparations, comprising:

(a) (i) a “rough-”, “incomplete-” or medium-filtered microbiota-comprising fecal sample or isolate comprising or retaining its native or wild type physiological components or nutritive agents for bacteria of the microbiota,

wherein optionally the sample or isolate can pass through an about 0.1 mm sieve opening or filter hole; or

(ii) a “rough-”, “incomplete-” or medium-filtered microbiota-comprising fecal sample or isolate made by a process comprising; (1) providing a fecal material; and (2) passing the fecal material an about 0.1 mm sieve opening or filter hole;

(b) the “rough-”, “incomplete-” or medium-filtered microbiota-comprising fecal sample or isolate of (a), wherein the fecal sample or isolate consists of a human fecal material;

(c) the “rough-”, “incomplete-” or medium-filtered microbiota-comprising fecal sample or isolate of (a) or (b), further comprising by having added: a fiber, biologically active proteins or peptides, micronutrients, fats, sugars or small carbohydrates, trace elements, mineral salts, ash, mucous, amino acids, nutrients, vitamins or minerals, or all or any combination thereof, optionally “added back” to, reconstitute a “wild type” healthy flora or human microbiota environment;

(d) the liquid preparation of any of (a) to (c), further processed or formulated for either freezing or freeze-drying into a powder, or equivalent, and optionally further comprising a cryoprotectant, a lyoprotectant, or a preservative; or

(f) the liquid preparation of (d), further processed or formulated by reconstituting the frozen or freeze-dried powder in a liquid, wherein optionally the liquid is a sterile saline.

In alternative embodiments, the invention provides formulations or pharmaceutical preparations comprising:

(a) the formulation or pharmaceutical preparation of the invention, wherein the bacteria or microbiota component has been cultured (or placed into an enrichment culture), or cultured (or placed into an enrichment culture) under anaerobic conditions, or harvested, stored and/or cultured (or placed into an enrichment culture) under anaerobic conditions,

wherein optionally the highly filtered or substantially purified microbiota and liquid preparation or formulation of the invention is cultured or placed into an enrichment culture, or optionally a highly filtered or substantially purified microbiota is cultured before addition of the liquid preparation or formulation of the invention, or optionally the highly filtered or substantially purified microbiota is cultured or placed into an enrichment culture before and after addition of the liquid preparation or formulation of the invention;

(b) the formulation or pharmaceutical preparation of (a), wherein the bacteria or microbiota component is cultured for about 2 to about 72 hours (hrs), or about 1 hour to 24 hours, or about 30 minutes to 12 hours, to increase the numbers of the bacteria and their products without needing to use larger numbers of donors;

(c) the formulation or pharmaceutical preparation of (a) or (b), wherein the bacteria or microbiota component is cultured or incubated in a liquid enrichment culture medium in aerobic or in anaerobic conditions using appropriate nutrient broths; or

(d) the formulation or pharmaceutical preparation of any of (a) to (c), wherein the cultured bacteria or microbiota component is aliquotted and frozen or freeze-dried or lyophilized or cryodesiccated.

In alternative embodiments, formulations or pharmaceutical preparations of the invention further comprise, or have added to: at least one bacteria or species of a Firmicutes, Bacteroidetes, a Bacillus, or a Bacillus thurigiensis, wherein optionally the Firmicutes, Bacteroidetes, a Bacillus is from a culture.

In alternative embodiments, formulations or pharmaceutical preparations of the invention further comprise, or have added to: at least one probiotic or prebiotic,

wherein optionally the prebiotic comprises an inulin, lactulose, extracts of artichoke, chicory root, oats, barley, various legumes, garlic, kale, beans or flacks or an herb,

wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E. coli, a Strep fecalis and equivalents.

In alternative embodiments, formulations or pharmaceutical preparations of the invention further comprise, or have added to: at least one congealing agent, wherein optionally the congealing agent comprises an arrowroot or a plant starch, a powdered flour, a powdered potato or potato starch, an absorbant polymer, an Absorbable Modified Polymer (AMP®), EndoClot, Santa Clara, Calif.), and/or a corn flour or a corn starch.

In alternative embodiments, formulations or pharmaceutical preparations of the invention further comprise, or have added to: at least one an anti-inflammatory agent, wherein optionally the inflammatory agent comprises or is a 4 or a 5-amino-salicylate, an olsalazine (e.g., DIPENTUM™), a mesalazine (also known as mesalamine or a 5-aminosalicylic acid (5-ASA), e.g., ASACOL™ or LIALDA™), a sulfasalazine (e.g., AZULFIDINE™, SALAZOPYRIN™ or SULAZINE™), and/or a balsalazide (e.g. COLAZAL™ or COLAZIDE™), or an equivalent thereof or a combination thereof.

In alternative embodiments, formulations or pharmaceutical preparations of the invention further comprise, or have added to: at least one opiate inhibitor or opiate antagonist, wherein optionally the opiate inhibitor or opiate antagonist is a methylnaltrexone bromide, a naltrexone (e.g., REVIA™, DEPADE™, VIVITROL™), or a nalmefene glucuronide.

In alternative embodiments, formulations or pharmaceutical preparations of the invention further comprise, or have added to: at least one acid suppressant, antacid and/or proton pump inhibitor, wherein optionally the acid suppressant is an H2 Receptor Antagonist, wherein optionally the H2 Receptor Antagonist is a cimetidine (e.g., TAGAMET™), a ranitidine (e.g., ZANTAC™), or an equivalent, wherein optionally the Proton Pump Inhibitor is an omeprazole (e.g., LOSEC™, ANTRA™, GASTROLOC™, MOPRAL™, OMEPRAL™, PRILOSEC™), an esameprazole (e.g., NEXIUM™), a pantoprazole (e.g., SOMAC™, TECTA™, PANTOLOC™, PROTIUM™ PROTONIX™) and equivalents.

In alternative embodiments, formulations or pharmaceutical preparations of the invention further comprise, or have added to: an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a probiotic or a prebiotic nutrient.

In alternative embodiments, formulations or pharmaceutical preparations of the invention further comprise, or have added to: at least one Biofilm Disrupting Compound, wherein optionally the biofilm disrupting compound comprises an enzyme, a deoxyribonuclease (DNase), N-acetylcysteine, an alginate lyase, glycoside hydrolase dispersin B; Quorum-sensing inhibitors e.g., ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.

In alternative embodiments, a formulation or pharmaceutical preparation of the invention is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, such as EUDRAGIT ST™ (Evonik Industries AG, Essen, Germany), which dissolves at pH 7 or greater, e.g., comprises a multimatrix (MMX) formulation.

In alternative embodiments, the invention provides a delivery vehicle, product of manufacture, container, syringe, device or bag, comprising: a formulation or pharmaceutical preparation of the invention.

In alternative embodiments, the invention provides a delivery vehicle, formulation, composition, pharmaceutical preparation, product of manufacture, container, bag or device comprising: a formulation or pharmaceutical preparation of the invention, initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

In alternative embodiments, the invention provides methods for the amelioration, stabilization, treatment and/or prevention of an infection, disease, treatment, poisoning or a condition having a bowel dysfunction component or side-effect comprising administering to an individual in need thereof via a delivery vehicle, formulation, composition, pharmaceutical preparation, product of manufacture, container or device comprising: a formulation or pharmaceutical preparation of the invention. In alternative embodiments, the infection, disease, treatment, poisoning or condition having a bowel dysfunction component or side-effect comprises a constipation, an inflammatory bowel disease (IBD), Crohn's disease, hepatic encephalopathy, enteritis, colitis, irritable bowel syndrome (IBS), fibromyalgia (FM), chronic fatigue syndrome (CFS), depression, attention deficit/hyperactivity disorder (ADHD), multiple sclerosis (MS), systemic lupus erythematosus (SLE), travelers' diarrhea, small intestinal bacterial overgrowth, chronic pancreatitis, a pancreatic insufficiency, exposure to a poison or a toxin or for an infection, a toxin-mediated traveler's diarrhea, a poisoning, a pseudomembranous colitis, a Clostridium infection, a C. perfringens welchii or a Clostridium difficile infection, a neurological condition, Parkinson's disease, myoclonus dystonia, autism, amyotrophic lateral sclerosis or multiple sclerosis, Grand mal seizures or petit mal seizures, a halitosis, a hepato-renal syndrome and/or a diverticulitis or a recurrent diverticulitis, an atopic condition, an asthma, an Attention Deficit Disorder (ADD and ADHD), an obsessive compulsive disorder (OCD), a depression, a schizophrenia and/or a mood disorder.

In alternative embodiments, the invention provides methods for the amelioration, stabilization, treatment and/or prevention of, or decreasing or delaying the symptoms of, an infection, disease, treatment, poisoning or a condition having a bowel dysfunction component or side-effect, or for the amelioration, treatment and/or prevention of a constipation, for the treatment of an abdominal pain, a non-specific abdominal pain or a diarrhea, a diarrhea caused by: a drug side effect or a psychological condition or Crohn's Disease, a poison, a toxin or an infection, a toxin-mediated traveller's diarrhea, or a Clostridium or a C. perfringens welchii or a C. difficile infection or a pseudo-membranous colitis associated with a Clostridium infection, or for preventing, or decreasing or delaying the symptoms of, or ameliorating or treating individuals with spondyloarthropathy, spondylarthritis or sacrolileitis (an inflammation of one or both sacroiliac joints); a nephritis syndrome; an inflammatory or an autoimmune condition having a gut or an intestinal component; lupus; irritable bowel syndrome (IBS or spastic colon); or a colitis; Ulcerative Colitis or Crohn's Colitis; constipation; autism; a degenerative neurological diseases; amyotrophic lateral sclerosis (ALS), Multiple Sclerosis (MS) or Parkinson's Disease (PD); a Myoclonus Dystonia; Steinert's disease; proximal myotonic myopathy; an autoimmune disease; Rheumatoid Arthritis (RA) or juvenile idiopathic arthritis (HA); Chronic Fatigue Syndrome; benign myalgic encephalomyelitis; chronic fatigue immune dysfunction syndrome; chronic infectious mononucleosis; epidemic myalgic encephalomyelitis; obesity; hypoglycemia, pre-diabetic syndrome, type I diabetes or type II diabetes; Idiopathic thrombocytopenic purpura (ITP); an acute or chronic allergic reaction; hives, a rash, a urticaria or a chronic urticaria; and/or insomnia or chronic insomnia, Grand mal seizures or petit mal seizures, a halitosis, a hepato-renal syndrome and/or a diverticulitis or a recurrent diverticulitis, an atopic condition, an asthma, an Attention Deficit Disorder (ADD and ADHD), an obsessive compulsive disorder (OCD), a depression, a schizophrenia and/or a mood disorder, comprising:

administering to an individual in need thereof via a formulation or pharmaceutical preparation of the invention, in single, repeat or multiple administrations, deliveries or infusions.

In alternative embodiments, the invention provides uses of a formulation or pharmaceutical preparation of the invention, in the preparation of a medicament for the amelioration, stabilization, treatment and/or prevention of an infection, disease, treatment, poisoning or a condition having a bowel dysfunction component or side-effect, or for the amelioration; treatment and/or prevention of a constipation, for the treatment of an abdominal pain, a non-specific abdominal pain or a diarrhea, a diarrhea caused by: a drug side effect or a psychological condition or Crohn's Disease, a poison, a toxin or an infection, a toxin-mediated traveler's diarrhea, or a Clostridium or a C. perfringens welchii or a C. difficile infection or a pseudo-membranous colitis associated with a Clostridium infection.

In alternative embodiments, the invention provides uses of a formulation or pharmaceutical preparation of the invention, in the preparation of a medicament for: preventing, decreasing the symptoms of, ameliorating, stabilizing, or treating: spondyloarthropathy, spondylarthritis or sacrolileitis (an inflammation of one or both sacroiliac joints); a nephritis syndrome; an inflammatory or an autoimmune condition having a gut or an intestinal component such as lupus, irritable bowel syndrome (IBS or spastic colon) or a colitis such as Ulcerative Colitis or Crohn's Colitis; constipation, autism; a degenerative neurological diseases such as amyotrophic lateral sclerosis (ALS), Multiple Sclerosis (MS) or Parkinson's Disease (PD); a Myoclonus Dystonia (e.g., Steinert's disease or proximal myotonic myopathy); an autoimmune disease such as Rheumatoid Arthritis (RA) or juvenile idiopathic arthritis (JIA); Chronic Fatigue Syndrome (including benign myalgic encephalomyelitis, chronic fatigue immune dysfunction syndrome, chronic infectious mononucleosis, epidemic myalgic encephalomyelitis); obesity; hypoglycemia, pre-diabetic syndrome, type I diabetes or type II diabetes; Idiopathic thrombocytopenic purpura (ITP); an acute or chronic allergic reaction such as hives, a rash, a urticaria or a chronic urticaria; and/or insomnia or chronic insomnia, Grand mal seizures orpetit mal seizures, halitosis, hepato-renal syndrome and/or diverticulitis or a recurrent diverticulitis, an atopic condition, an asthma, an Attention Deficit Disorder (ADD and ADHD), an obsessive compulsive disorder (OCD), a depression, a schizophrenia and/or a mood disorder.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In alternative embodiments, the invention provides compositions, e.g., formulations and pharmaceutical preparations, products of manufacture, and containers and delivery vehicles, and devices and delivery materials, comprising treated and/or isolated human GI microbiota for Fecal Microbiota Transplantation (FMT) (previously known as “Fecal Bacteriotherapy”). In alternative embodiments, formulations or pharmaceutical preparations of the invention are designed or formulated for implantation of living bacteria, or delivery of an active ingredient (e.g., a liquid preparation of the invention) into the distal small bowel and/or the colon.

In alternative embodiments, the invention provides compositions and methods comprising use of both bacterial cells, e.g., a partial or a complete representation of the human GI microbiota, and an isolated, processed, filtered, concentrated, reconstituted and/or artificial liquid component of the flora (the microbiota) which comprises, among others ingredients, bacterial secretory products such as e.g., bacteriocins (proteinaceous toxins produced by bacteria, including colicin, troudulixine or putaindicine, or microcin or subtilosin A), lanbiotics (a class of peptide antibiotics that contain a characteristic polycyclic thioether amino acid lanthionine or methyllanthionine, and unsaturated amino acids dehydroalanine and 2-aminoisobutyric acid; which include thuricin (which is secreted by bacilli in donor stools), nisin, subtilin, epidermin, mutacin, mersacidin, actagardine, cinnamycin), a lacticin (a family of pore-forming peptidic toxins) and other antimicrobial or anti-inflammatory compounds and/or biologically active molecules (BAMs) produced by bacteria or other microorganisms of the microbiota, and/or which are found in the “liquid component” of the microbiota. This “liquid component” of the invention is missing in the “cells only” Fecal Microbiota Transplantation (FMT) products, e.g., filtered FMT, described to date.

This embodiment of the invention comprising a “liquid component” can be safer and/or more effective, e.g., can be “customized” for the treatment of a particular condition, infection or disease, as compared to the implantation (e.g., transplantation) of crude homogenised human stool, which has been very successful in the cure of conditions such as chronic Clostridium difficile infection of the gut flora. CDI, particularly the relapsing forms, has been difficult to cure with antibiotics, but have been cured in over 90% of patients implanted with crude “wild type” bacteria from normal flora collected from a donor and infused in toto into a recipient, see e.g., Khoruts; et al. WO 2012 122478. Compositions and methods of the invention comprising a reconstituted or co-administered “liquid component” can have equal or great efficacy.

Use of FMT in other conditions, for example, to treat a colitis, has required more aggressive and repeated crude “wild type” bacteria infusions, indicating that there are factors operating in such conditions that are different from the relatively easy cure of CDI with one or two FMT infusions. The embodiment of the invention comprising use of a “liquid component”, e.g., a component with more than the cellular components of the microbiome, are effective where “cellular only” FMT formulations fail or lack sufficient efficacy. In alternative embodiments, the non-cellular “liquid component” of the invention, for example, secreted, excreted or otherwise liquid components or the microbiota, e.g., biologically active molecules (BAMs), which can be antibiotics or anti-inflammatories, are preserved, retained or reconstituted in a flora extract, or a formulation of the invention together with the bacteriophages which will pass through the 22 micron filter described above. Thus, in alternative embodiments, non-cellular “liquid components” of the invention can help with the inflammatory processes, e.g., as types of bacterially-derived anti-inflammatory components of normal flora and have healthy bacteriophage anti-bacterial activity. In alternative embodiments, this non-cellular “liquid component” is specifically preserved, filtered, reconstituted and/or recreated (e.g., synthetic) and is kept in a composition of the invention, or is or added to or administered with a composition of the invention.

In alternative embodiments, the current invention provides improved compositions and methods of putting together, formulating or reconstituting complete or substantially complete extracted flora, by also comprising, reconstituting or adding specific additions, e.g., a non-cellular “liquid component” and/or components thereof, to improve on the efficacy, performance and/or safety of the “cellular” component. In alternative embodiments, the compositions and methods of the invention provide not only improved functionality but also new applications to various conditions not previously attended to.

In alternative embodiments, the compositions and methods of the invention also provide for the disruption of biological films, e.g., biofilms, to improve implantation characteristics and have greater efficacy in reversing conditions.

In alternative embodiments, the compositions and methods of the invention comprising use of FMT therapy comprising a non-cellular “liquid component” and/or components thereof are used for conditions such as an autoimmune disease (e.g., an autoimmune colitis such as Ulcerative Colitis (UC), a multiple sclerosis (MS)), or an autism, and others. In alternative embodiments, the compositions and methods of the invention are used to treat, ameliorate, reverse or cure diseases, infections or conditions which are not treatable or curable with one or two infusions (as with CDI), but rather need prolonged administration to achieve cure or a maintenance therapy to maintain remission, e.g. Ulcerative Colitis (UC). Alternatively, the embodiment comprising a cellular and a liquid component composition, e.g., as the description of (3), above, can be used here to also implant (e.g., administer to a patient) the missing (in the patient) flora components seen in a health, or “wild type” (WT) individual. In alternative embodiments, the liquid component has a powerful anti-spore activity.

In alternative embodiments, the compositions and methods of the invention can be formulated in any solid or liquid form, e.g., as a pharmaceutical, food or supplement formulation, e.g., an encapsulated preparation and/or a powdered yoghurt preparation (which can be encapsulated) to e.g., gain acceptance and for prolonged use. In alternative embodiments, the compositions and methods of the invention can be formulated as enemas. In alternative embodiments, the compositions and methods of the invention can be formulated as an oral product, e.g., in an encapsulated form. In alternative embodiments, compositions and methods of the invention comprises use of formulations comprising all the stool components, i.e., comprising a non-cellular “liquid component”, or reconstituted or synthetic equivalent thereof, and not just the bacterial cellular component.

In alternative embodiments, the compositions of the invention comprise various biologically active molecules (BAMs), including anti-inflammatory components of the flora, or microbiota. In alternative embodiments, the liquid or dissolved components are included in a cellular preparation or are added back to the cells, e.g., when the stool is filtered down to its cellular mass alone. In alternative embodiments, the compositions of the invention comprise a representation of a cellular microbiota, e.g., a complete or substantially complete human microbiota, further comprising added components or “add backs” put into the compositions so as to give further utility, better efficacy and/or improved safety in the varying applications. For example, in alternative embodiments, biologically active molecules (BAM's) that are “added back”, including bacterial secretory products, anti-inflammatory reagents or other compositions (e.g., as secreted by the “host” bowel, e.g., interleukins, cytokines, leukotrienes, eicosanoids and the like), antibodies (e.g., IgAs, IgGs, IgMs, antigen-binding antibody fragments or synthetic antibody-like peptides or reagents), prebiotics, probiotics, anti-biofilm reagents or biofilm-dissolving reagents, and/or antimicrobials, antibiotics or antifungal agents. In alternative embodiments, these or other “added back” components can be man-made or pure or crude or concentrated non-cellular “liquid component” of a microbiota, e.g., such as bacterial secretory products such as: thuricin (which is secreted by bacilli in donor stools), bacteriocins (proteinaceous toxins produced by bacteria, including colicin, troudulixine or putaindicine, or microcin or subtilosin A), lanbiotics (including a nisin, a subtilin, an epidermin, a mutacin, a mersacidin, an actagardine and/or a cinnamycin), a lacticin or related pore-forming peptidic toxin, and/or other antimicrobial or anti-inflammatory compounds and/or biologically active molecules (BAMs) produced by bacteria or other microorganisms of the microbiota. In alternative embodiments, one or all of these, or other, additives are added to compositions of the invention, e.g., “added back” to such the final formulation closely or better simulates or has the same properties of or is a substantial representation of a normal or wild type human microbiota.

In alternative embodiments, prior to treatment with the extracted flora, the physician may also choose to use a purgative to reduce the volume of the flora so that implantation is easier in an empty bowel.

### Additional Optional Ingredients

Congealing Agents

In alternative embodiments, following infusion of a composition of the invention (including any microbiota- or FMT-comprising composition of the invention), e.g., following a transcolonoscopic infusion or infusion by an enema (particularly into the colon of patients with Ulcerative Colitis), or following an oral administration, a congealing agent is also used or administered. For example, when a patient is wheeled into recovery the infused liquid moves down to the sigmoid colon, then into the rectum; this causes the patient to feel extreme urgency to the extent that many patients cannot hold the liquid and suffer fecal incontinence while in bed or while being transported to the bathroom. To prevent this, the invention includes the use of added congealing agents, e.g., including arrowroot or a plant starch, e.g., a powdered flour, a powdered potato or potato starch, an absorbant polymer (e.g., an Absorbable Modified Polymer (AMP®), EndoClot, Santa Clara, Calif.), and/or a corn flour or corn starch. This absorbs water rapidly, produces a gelled matrix and keeps the infused microbiota congealed for hours in the cecum preventing the progression to the rectum.

Probiotics and Prebiotics

In alternative embodiments, additives that are also included in a composition of the invention (e.g., the liquid preparation embodiment, the highly filtered or substantially purified microbiota and liquid preparation mix, or the “rough-”, “incomplete-” or medium-filtered microbiota-comprising fecal sample, and/or the cultured microbiota embodiment), or a composition used to practice the invention, includes one or more prebiotics such as inulin, lactulose, extracts of artichoke, chicory root, oats, barley, various legumes, garlic, kale, beans or flacks and at times prebiotics may include herbs.

In alternative embodiments, additives may include flora components such as Bacteroidetes, Firmicutes, Bacillus (e.g., Bacillus thurigiensis) or any combination thereof. In alternative embodiments, cultured components are back to the flora to fortify or expand specific genus or species, e.g., Bacteroidetes, Firmicutes, Bacillus or Bacillus thurigiensis. Probiotics may at times be included as single cultured components. They would avoid multiply cultured components as they lose their implantation characteristics.

Antibiotics, Antimicrobials

In alternative embodiments, antibiotics and/or other antimicrobials are included in a composition of the invention, e.g., added back to a liquid formulation or preparation of the invention, or cell preparation of the invention. In alternative embodiments, the antimicrobial or antibiotic is or comprises one or more of a: glycopeptide antibiotic, wherein optionally the glycopeptide antibiotic is a vancomycin, a teicoplanin (e.g., TARGOCID™), a telavancin (e.g., VIBATIV™), a bleomycin (e.g., BLENOXANE™), a ramoplanin or a decaplanin; or, a fidaxomycin, a gentamycin, a neomycin, a streptomycin, a paromomycin, a kanamycin, a rifaximin (e.g., the extended intestinal release (EIR) rifaximin) or another rifamycin (including e.g., the rifamycin derivatives rifampicin (or rifampin), rifabutin, rifapentine and rifalazil), or an ansamycin, a geldanamycin, an ansamitocin, or an anti-protozoal agent such as nitazoxanide (e.g., DAXON™, DEXIDEX™, KIDONAX™, MITAFAR™, PACOVANTON™, PARAMIX™), a furazolidone (e.g., FUROXONE™, DEPENDAL-M™), a nitroimidazole or metronidazole (e.g., a 5-nitroimidazole, FLAGYL™), a nifuroxazide (e.g., AMBATROL™, ANTINAL™, BACIFURANE™, DIAFURYL™) or a bismuth (e.g., bismuth subsalicylate), also including various groups of antibiotics such as a penicillin (e.g., penicillin G, procaine penicillin, benzathine penicillin or penicillin V), a macrolide (e.g., erythromycin, a clarithromycin, a dirithromycin (e.g., DYNABAC™), a roxithromycin (e.g., XTHROCIN™, ROXL-150™, ROXO™, SURLID™), a telithromycin (e.g., KETEK™) or an azithromycin such a ZITHROMAX™, AZITHROCIN™), a tetracycline, a cephalosporin, a carbapenem (e.g., imipenem, a meropenem such as MONAN™, MERONEM™), a monobactam, a lincosamide or a clindamycin (e.g., DALACIN™), a quinolone (e.g., a fluoroquinolone) and/or a sulphonamide, a fradicin (e.g., NEOBIOTIC™), or an equivalent thereof or a combination thereof.

In alternative embodiments, the antimicrobial or antibiotic is or comprises one or more of: an aminoglycoside antibiotic (e.g., a gentamycin, a neomycin, a streptomycin, a paromomycin and/or a kanamycin), amphenicol, ansamycin, beta-lactam (β-lactam), carbapenem, cephalosporin, cephamycin, monobactam, oxacephem, a lincosamide antibiotic (e.g., clindamycin, lincomycin), a macrolide antibiotic (e.g., an azithromycin, clarithromycin, dirithromycin, erythromycin), glycopeptide antibiotic (e.g., a vancomycin, teicoplanin, telavancin, bleomycin, ramoplanin and/or a decaplanin), a polypeptide antibiotic (e.g., actinomycin, such as actinomycin D; bacitracin; bacitracin), tetracycline, or a 2,4-diaminopyrimidine class antibiotic, a clavacin (also known as clairformin, claviform, expansine, clavatin, expansin, gigantin, leucopin, patuline or patulin), or an equivalent thereof or a combination thereof.

In alternative embodiments, methods of the invention comprise pre-treatment, co-treatment (simultaneous treatment) and/or post-treatment with an antibiotic and/or other antimicrobial, including e.g., Vancomycin, Rifaximin, Metronidazole, Rifampicin or any class of antibiotics to suppress the particular pathogen or pathogens, e.g., that are being treated.

Preservatives, Cryoprotectants, Lyoprotectants

In alternative embodiments, to any composition of the invention (e.g., the liquid preparation embodiment, the highly filtered or substantially purified microbiota and liquid preparation mix, or the “rough-”, “incomplete-” or medium-filtered microbiota-comprising fecal sample, and/or the cultured microbiota embodiment) may be added various preservatives, cryoprotectants and/or lyoprotectants, including e.g., various polysaccharides or sugars (such as sucrose, fructose, lactose, mannitol), glycerol, polyethylene glycol (PEG), trehalose, glycine, glucose, dextran and/or erythritol. In alternative embodiments, other cryoprotectants that can be used are ethylene glycol, 1,2-Propanediol, Methylcelliosolve, Dimethyl Formamide, or Dimethylsulphoxide Methanol. In alternative embodiments the content of these cryoprotectants are between about 1% and about 50% but generally between about 5% and about 15% is adequate.

Because of the ability to freeze and/or freeze-dry, or spray dry, any composition of the invention, in alternative embodiments there are different types of final products that can be manufactured. In alternative embodiments a product or formulation of the invention is a liquid and can be used fresh as an enema. In alternative embodiments a product or formulation of the invention is frozen and kept at e.g. minus 80 degrees for usage later given a cryoprotectant is added.

Biofilm Disrupting Compounds

In alternative embodiments, biofilm disrupting compounds added into a composition or formulation of the invention (e.g., the liquid preparation embodiment, the highly filtered or substantially purified microbiota and liquid preparation mix, or the “rough-”, “incomplete-” or medium-filtered microbiota-comprising fecal sample, and/or the cultured microbiota embodiment), or used to practice a method of the invention. In alternative embodiments, in practicing the methods of the invention, biofilm disrupting compounds are administered before or during (co-administered), or co-formulated with (e.g., in a multilaminated tablet or capsule), or separately formulated, as the administered composition or formulation of the invention. In alternative embodiments, disrupting biofilms are used to separate from the colonic mucosa an adherent polysaccharide/DNA-containing layer, the so-called “biofilm”, to achieve a mucosa which can better accept implantation of incoming wild-type and/or cultured flora components and compositions of the invention.

In alternative embodiments, other biofilm disrupting components or agents also can be used, e.g., enzymes such as deoxyribonuclease (DNase), N-acetylcysteine, alginate lyase, glycoside hydrolase dispersin B; Quorum-sensing inhibitors e.g., ribonucleic acid III inhibiting peptide, Salvadora persica extracts, Competence-stimulating peptide, Patulin and penicillic acid; peptides—cathelicidin-derived peptides, small lytic peptide, PTP-7 (a small lytic peptide, see e.g., Kharidia (2011) J. Microbiol. 49(4):663-8, Epub 2011 Sep. 2), Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones and/or macrolide antibiotics or any combination thereof.

In alternative embodiments, biofilm disrupting components or agents are administered before and during the administration of a composition of this invention, e.g., as an FMT, in whatever format or formulation this may take place, for example, as a capsule.

In alternative embodiments, biofilm disrupting agents are added either before treatment and/or during and/or after treatment with a composition of the invention. In alternative embodiments, biofilm disrupting agents are, used singly or in combination.

In alternative embodiments, biofilm disrupting agents include particular enzymes and degrading substances including in N-acetylcysteine, deoxyribonuclease (DNase). Others would include Alginate, lyase and Glycoside hydrolase dispersin, Ribonucleic-acid-III inhibiting peptide (RIP), Salvadora persica extracts, Competence-stimulating peptide (CSP) Patulin (PAT) and penicillic acid (PA)/EDTA, Cathelicidin-derived peptides, Small lytic peptide, PTP-7, Nitric oxide, Chlorhexidine, Povidone-iodine (PI), Nanoemulsions, Lytic bacteriophages, Lactoferrin/xylitol hydrogel, Synthetic iron chelators, Cranberry components, Curcumin, Acetyl-11-keto-boswellic acid (AKBA), Barley coffee (BC) components, silver nanoparticles, azithromycin, clarithromycin, gentamicin, streptomycin and also Disodium EDTA. Ozone insufflations of the colon can also be used to disrupt the biofilm.

### Unit Dosage Forms and Formulations, Foods, and Delivery Vehicles

In alternative embodiments, following filtration, a composition of the invention (e.g., the liquid preparation embodiment, the highly filtered or substantially purified microbiota and liquid preparation mix, or the “rough-”, “incomplete-” or medium-filtered microbiota-comprising fecal sample, and/or the cultured microbiota embodiment) can be further processed by, e.g., spray-drying or equivalent, e.g., spray-drying in an inert gas or freeze-drying under similar conditions, thus ending up with a powdered product. In alternative embodiments, a composition is manufactured, labelled or formulated as a liquid, a suspension, a spray, a gel, a geltab, a semisolid, a tablet, or sachet, a capsule, a lozenge, a chewable or suckable unit dosage form, or any pharmaceutically acceptable formulation or preparation. In alternative embodiments, a composition of the invention is incorporated into a food or a drink (e.g., a yogurt, ice cream, smoothie), a feed, a nutritional or a food or feed supplement (e.g., liquid, semisolid or solid), and the like.

For example, a composition of the invention can be manufactured, labelled or formulated as an orally disintegrating tablet as described e.g., in U.S. Pat. App. Publication No. 20100297031. A composition of the invention can be a polyol/thickened oil suspension as described in U.S. Pat. Nos. 6,979,674; 6,245,740. A composition of the invention can be encapsulated, e.g., encapsulated in a glassy matrix as described e.g., in U.S. Pat. App. Publication No. 20100289164; and U.S. Pat. No. 7,799,341. A composition of the invention can be manufactured, labeled or formulated as an excipient particle, e.g., comprising a cellulosic material such as microcrystalline cellulose in intimate association with silicon dioxide, a disintegrant and a polyol, sugar or a polyol/sugar blend as described e.g.; in U.S. Pat. App. Publication No. 20100285164. A composition of the invention can be manufactured, labeled or formulated as an orally disintegrating tablet as described e.g., in U.S. Pat. App. Publication No. 20100278930. A composition of the invention can be manufactured, labeled or formulated as a spherical particle, as described e.g., in U.S. Pat. App. Publication No. 20100247665, e.g., comprising a crystalline cellulose and/or powdered cellulose. A composition of the invention can be manufactured, labeled or formulated as a rapidly disintegrating solid preparation useful e.g. as an orally-disintegrating solid preparation, as described e.g., in U.S. Pat. App. Publication No. 20100233278. A composition of the invention can be manufactured, labeled or formulated as a solid preparation for oral application comprising a gum tragacanth and a polyphosphoric acid or salt thereof, as described e.g., in U.S. Pat. App. Publication No. 20100226866.

A composition of the invention can be manufactured, labeled or formulated using a water soluble polyhydroxy compound, hydroxy carboxylic acid and/or polyhydroxy carboxylic acid, as described e.g., in U.S. Pat. App. Publication No. 20100222311. A composition of the invention can be manufactured, labeled or formulated as a lozenge, or a chewable and suckable tablet or other unit dosage form, as described e.g., in U.S. Pat. App. Publication No. 20100184785.

A composition of the invention can be manufactured, labeled or formulated in the form of an agglomerate, as described e.g., in U.S. Pat. App. Publication No. 20100178349. A composition of the invention can be manufactured, labeled or formulated in the form of a gel or paste, as described e.g., in U.S. Pat. App. Publication No. 20060275223. A composition of the invention can be manufactured, labeled or formulated in the form of a soft capsule, as described e.g., in U.S. Pat. No. 7,846,475, or U.S. Pat. No. 7,763,276.

The polyols used in compositions of the invention can be micronized polyols, e.g., micronized polyols, e.g., as described e.g., in U.S. Pat. App. Publication No. 20100255307, e.g., having a particle size distribution (d₅₀) of from 20 to 60 μm, and a flowability below or equal to 5 s/100 g, or below 5 s/100 g.

Gradual or Delayed Release Formulations

In alternative embodiments, the invention provides compositions formulated for delayed or gradual enteric release comprising at least one active agent (e.g., a formulation or pharmaceutical preparation of the invention) formulated with a delayed release composition or formulation, coating or encapsulation. In alternative embodiments, formulations or pharmaceutical preparations of the invention are designed or formulated for implantation of living bacteria, or delivery of active ingredient (e.g., a liquid preparation of the invention) into the distal small bowel and/or the colon. Thus, for this embodiment, it is important to allow the living bacteria to pass the areas of danger, e.g., stomach acid and pancreatic enzymes and bile, and reach undamaged to be viable and implant in the distal small bowel and especially the colon. In alternative embodiments, a formulation or pharmaceutical preparation of the invention is a liquid formulation, a microbiota-comprising formulation of the invention and/or a frozen or a freeze-dried version thereof. In alternative embodiments, preferably for the encapsulated format, all are in powdered form.

In alternative embodiments, compositions of the invention are formulated for delayed or gradual enteric release using cellulose acetate (CA) and polyethylene glycol (PEG), e.g., as described by Defang et al. (2005) Drug Develop. & Indust. Pharm. 31:677-685, who used CA and PEG with sodium carbonate in a wet granulation production process.

In alternative embodiments, compositions of the invention are formulated for delayed or gradual enteric release using a hydroxypropylmethylcellulose (HPMC), a microcrystalline cellulose (MCC) and magnesium stearate, as described e.g., in Huang et al. (2004) European J. of Pharm. & Biopharm. 58: 607-614).

In alternative embodiments, compositions of the invention are formulated for delayed or gradual enteric release using e.g., a poly(meth)acrylate, e.g. a methacrylic acid copolymer B, a methyl methacrylate and/or a methacrylic acid ester, a polyvinylpyrrolidone (PVP) or a PVP-K90 and a EUDRAGIT® RL PO™, as described e.g., in Kuksal et al. (2006) AAPS Pharm. 7(1), article 1, E1 to E9.

In alternative embodiments, compositions of the invention are formulated for delayed or gradual enteric release as described in U.S. Pat. App. Pub. 20100239667. In alternative embodiments, the composition comprises a solid inner layer sandwiched between two outer layers. The solid inner layer can comprise a formulation or pharmaceutical preparation of the invention and one or more disintegrants and/or exploding agents, one of more effervescent agents or a mixture. Each outer layer can comprise a substantially water soluble and/or crystalline polymer or a mixture of substantially water soluble and/or crystalline polymers, e.g., a polyglycol. These can be adjusted in an exemplary composition of the invention to achieve delivery of the living components of an FMT distally down the bowel.

In alternative embodiments, compositions of the invention are formulated for delayed or gradual enteric release as described in U.S. Pat. App. Pub. 20120183612, which describes stable pharmaceutical formulations comprising active agents in a non-swellable diffusion matrix. In alternative embodiments, a formulation or pharmaceutical preparation of the invention is released from a matrix in a sustained, invariant and, if several active agents are present, independent manner and the matrix is determined with respect to its substantial release characteristics by ethylcellulose and at least one fatty alcohol to deliver bacteria distally.

In alternative embodiments, a formulation or pharmaceutical preparation of the invention is formulated for delayed or gradual enteric release as described in U.S. Pat. No. 6,284,274, which describes a bilayer tablet containing an active agent (e.g., an opiate analgesic), a polyalkylene oxide, a polyvinylpyrrolidone and a lubricant in the first layer and a second osmotic push layer containing polyethylene oxide or carboxymethylcellulose.

In alternative embodiments, a formulation or pharmaceutical preparation of the invention is formulated for delayed or gradual enteric release as described in U.S. Pat. App. Pub. No. 20030092724, which describes sustained release dosage forms in which a nonopioid analgesic and opioid analgesic are combined in a sustained release layer and in an immediate release layer, sustained release formulations comprising microcrystalline cellulose, EUDRAGIT RSPO™, CAB-O-SIL™, sodium lauryl sulfate, povidone and magnesium stearate.

In alternative embodiments, a formulation or pharmaceutical preparation of the invention is formulated for delayed or gradual enteric release as described in U.S. Pat. App. Pub. 20080299197, describing a multi-layered tablet for a triple combination release of active agents to an environment of use, e.g., in the GI tract. In alternative embodiments, a multi-layered tablet is used, and it can comprise two external drug-containing layers in stacked arrangement with respect to and on opposite sides of an oral dosage form that provides a triple combination release of at least one active agent. In one embodiment the dosage form is an osmotic device, or a gastro-resistant coated core, or a matrix tablet, or a hard capsule. In these alternative embodiments, the external layers may contain biofilm dissolving agents and internal layers the living bacteria.

In alternative embodiments, a formulation or pharmaceutical preparation of the invention is formulated as multiple layer tablet forms, e.g., where a first layer provides an immediate release of a formulation or pharmaceutical preparation of the invention and a second layer provides a controlled-release of another (or the same) formulation or pharmaceutical preparation of the invention, or another active agent, as described e.g., in U.S. Pat. No. 6,514,531 (disclosing a coated trilayer immediate/prolonged release tablet), U.S. Pat. No. 6,087,386 (disclosing a trilayer tablet), U.S. Pat. No. 5,213,807 (disclosing an oral trilayer tablet with a core comprising an active agent and an intermediate coating comprising a substantially impervious/impermeable material to the passage of the first active agent), and U.S. Pat. No. 6,926,907 (disclosing a trilayer tablet that separates a first active agent contained in a film coat from a core comprising a controlled-release second active agent formulated using excipients which control the drug release, the film coat can be an enteric coating configured to delay the release of the active agent until the dosage form reaches an environment where the pH is above four).

In alternative embodiments, a formulation or pharmaceutical preparation of the invention is formulated for delayed or gradual enteric release as described in U.S. Pat. App. Pub. 20120064133, which describes a release-retarding matrix material such as: an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, polyvinylalcohols, polyvinylalcohol copolymers, polyethylene glycols, non-crosslinked polyvinylpyrrolidone, polyvinylacetates, polyvinylacetate copolymers or any combination. In alternative embodiments, spherical pellets are prepared using an extrusion/spheronization technique, of which many are well known in the pharmaceutical art. The pellets can comprise one or more formulations or pharmaceutical preparations of the invention, e.g., the liquid preparation embodiment, the highly filtered or substantially purified microbiota and liquid preparation mix, or the “rough-”, “incomplete-” or medium-filtered microbiota-comprising fecal sample, and/or the cultured microbiota embodiment, and can be designed or formulated for implantation into the distal small bowel and/or the colon.

In alternative embodiments, a formulation or pharmaceutical preparation of the invention is formulated for delayed or gradual enteric release as described in U.S. Pat. App. Pub. 20110218216, which describes an extended release pharmaceutical composition for oral administration, and uses a hydrophilic polymer, a hydrophobic material and a hydrophobic polymer or a mixture thereof, with a microenvironment pH modifier. The hydrophobic polymer can be ethylcellulose, cellulose acetate, cellulose propionate, cellulose butyrate, methacrylic acid-acrylic acid copolymers or a mixture thereof. The hydrophilic polymer can be polyvinylpyrrolidone, hydroxypropylcellulose, methylcellulose, hydroxypropylmethyl cellulose, polyethylene oxide, acrylic acid copolymers or a mixture thereof. The hydrophobic material can be a hydrogenated vegetable oil, hydrogenated castor oil, carnauba wax, candellia wax, beeswax, paraffin wax, stearic acid, glyceryl behenate, cetyl alcohol, cetostearyl alcohol or and a mixture thereof. The microenvironment pH modifier can be an inorganic acid, an amino acid, an organic acid or a mixture thereof. Alternatively, the microenvironment pH modifier can be lauric acid, myristic acid, acetic acid, benzoic acid, palmitic acid, stearic acid, oxalic acid, malonic acid, succinic acid, adipic acid, sebacic acid, fumaric acid, maleic acid; glycolic acid, lactic acid, malic acid, tartaric acid, citric acid, sodium dihydrogen citrate, gluconic acid, a salicylic acid, tosylic acid, mesylic acid or malic acid or a mixture thereof.

In alternative embodiments, a formulation or pharmaceutical preparation of the invention is a powder that can be included into a tablet or a suppository. In alternative embodiments, a formulation or pharmaceutical preparation of the invention can be a ‘powder for reconstitution’ as a liquid to be drunk placed down a naso-duodenal tube or used as an enema for patients to take home self-administer enemas for colitis for example. In alternative embodiments, a formulation or pharmaceutical preparation of the invention is micro-encapsulated, formed into tablets and/or placed into capsules, especially enteric-coated capsules.

In alternative embodiments, in practicing the methods of the invention, biofilm disrupting compounds are administered before or during (co-administered), or co-formulated with a composition or formulation of the invention. For example, in alternative embodiments, a composition or formulation of the invention and a biofilm disrupting compound (and/or any other alternative component of the invention, as discussed herein) are co-formulated, e.g., as multiple layer tablet form or as a multi-laminated tablet or capsule. In alternative embodiments of methods of the invention, biofilm disrupting compounds are separately formulated.

Feeds, Drinks, Candies, Nutritional or a Food or Feed Supplements

In alternative embodiments, a formulation or pharmaceutical preparation of the invention is incorporated into a food, a feed, a candy (e.g., a lollypop or a lozenge) a drink, a nutritional or a food or feed supplement (e.g., liquid, semisolid or solid), and the like, as described e.g., in U.S. Pat. App. Publication No. 20100178413. In one embodiment, a formulation or pharmaceutical preparation of the invention is incorporated into (manufactured as) a beverage as described e.g., in U.S. Pat. No. 7,815,956. For example, a composition of the invention is incorporated into a yogurt, an ice cream, a milk or milkshake, a “frosty”, “snow-cone”, or other ice-based mix, and the like.

In alternative embodiments, methods of the invention comprise pre-administration or co-administration of an acid inhibiting agent, e.g., an antacid, to facilitate implantation of the living bacteria of a composition of the invention, e.g., to facilitate administration or implantation of wild type microbiota and/or cultured bacteria of a composition of the invention. For example, in alternative embodiments, a composition or formulation of the invention and an acid inhibiting agent, e.g., an antacid, (and/or any other alternative component of the invention, as discussed herein) are co-formulated, e.g., as multiple layer tablet form or as a multi-laminated tablet or capsule. In alternative embodiments of methods of the invention, acid inhibiting agents are separately formulated.

In alternative embodiments, a formulation or pharmaceutical preparation of the invention is a freeze-dried powder form added to a food, e.g., a yogurt, an ice cream, a milk or milkshake, a “frosty”, “snow-cone”, or other ice-based mix, and the like. In one form of this invention it can be kept in a lid-storage (e.g., of a yogurt or ice cream) such that when it is twisted the powder falls into the product or formulation (e.g., yoghurt or ice cream) and then it can be stirred so as not to have the powder ferment ‘standing on the shelf’. Various flavourings can be added. In alternative embodiments, this is particularly important for administration of a composition of the invention, e.g., a wild type microbiota or a cultured bacteria, to a very young individual and/or a patient with autism or related disease or condition.

In alternative embodiments, these exemplary products are important when administered to babies who may have C. difficile or who may have acquired various pathogenic or abnormal bacteria, e.g., E. coli, Clostridia or Disulfovibrio, e.g., in autism.

### Methods of Use and Applications of Compositions of the Invention

In alternative embodiments, a formulation or pharmaceutical preparation of the invention, and/or a methods of the invention, or a use of the invention, is used to treat, ameliorate, prevent or reverse: a C. difficile infection, an Irritable Bowel Syndrome, an Inflammatory Bowel Disease such as Colitis and Crohn's metabolic syndrome, a diabetes type I and/or II, an obesity, a hepatic encephalopathy, a hepato-renal syndrome, an idiopathic constipation, a familial Mediterranean fever (FMF), gall stones (e.g., prevention of gall stone formation), a cancer, a colorectal cancer (e.g., prevention of colorectal cancer), and/or an acute gastrointestinal infection e.g., with a virus or a bacteria, or in traveller's diarrhoea. In alternative embodiments, a formulation or pharmaceutical preparation of the invention, and/or a methods of the invention, or a use of the invention, is used to treat, ameliorate, prevent or reverse: a halitosis, a hepato-renal syndrome and/or a diverticulitis, e.g., a recurrent diverticulitis.

In alternative embodiments, a product or formulation of the invention, and/or a methods of the invention, or a use of the invention, is used to treat, ameliorate, prevent or reverse: a non-specific abdominal pain, an idiopathic diarrhoea, an infection with a C. perfringens and/or a pseudo-membranous colitis.

In alternative embodiments, a product or formulation of the invention, and/or a methods of the invention, or a use of the invention, is used to treat, ameliorate, prevent or reverse: a non-gastrointestinal disorder, e.g., including a spondylo arthropathy, a spondylo arthritis, a sacro ileitis, a nephrotic syndrome.

In alternative embodiments, a product or formulation of the invention, and/or a methods of the invention, or a use of the invention, is used to treat, ameliorate, prevent or reverse: an auto-immune condition, e.g., such as a lupus, a rheumatoid arthritis, a chronic fatigue syndrome, an eczema, a fibromyalgia and/or other auto-immune conditions.

In alternative embodiments, a product or formulation of the invention, and/or a methods of the invention, or a use of the invention, is used to treat, ameliorate, prevent or reverse: a neurological disease or condition e.g., such as autism, amyotrophic lateral sclerosis (ALS), Multiple Sclerosis (MS), Parkinson's Disease (PD) and Myclonus Dystonia.

In alternative embodiments, a product or formulation of the invention, and/or a methods of the invention, or a use of the invention, is used to treat, ameliorate, prevent or reverse: an atopic conditions, an asthma, an Attention Deficit Disorder (ADD and ADHD), an obsessive compulsive disorder (OCD), a depression, a schizophrenia and/or a mood disorder.

In alternative embodiments, the invention provides compositions and methods for the amelioration, stabilization, treatment and/or prevention of an infection, disease, treatment, poisoning or a condition having a bowel dysfunction component or side-effect, or for the amelioration, stabilization, treatment and/or prevention of a constipation, for the treatment of an abdominal pain, a non-specific abdominal pain or a diarrhea, a diarrhea caused by: a drug side effect or a psychological condition or Crohn's Disease, a poison, a toxin or an infection, a toxin-mediated traveller's diarrhea, or a Clostridium or a C. difficile or a pseudo-membranous colitis associated with a Clostridium infection.

In alternative embodiments, the invention provides compositions and methods for the amelioration, stabilization, treatment and/or prevention of: a bowel dysfunction component or side-effect comprises an inflammatory bowel disease (IBD), Crohn's disease, hepatic encephalopathy, enteritis, colitis, Irritable Bowel Syndrome (IBS), fibromyalgia (FM), chronic fatigue syndrome (CFS), depression, attention deficit/hyperactivity disorder (ADHD), multiple sclerosis (MS), systemic lupus erythematosus (SLE), travellers' diarrhea, small intestinal bacterial overgrowth, chronic pancreatitis, apancreatic insufficiency, exposure to a poison or a toxin or for an infection, a toxin-mediated traveler's diarrhea, a poisoning, a pseudomembranous colitis, a Clostridium infection, a C. perfringens welchii or a Clostridium difficile infection, a neurological condition, Parkinson's disease, myoclonus dystonia, autism, amyotrophic lateral sclerosis, multiple sclerosis, Grand mal seizures or petit mal seizures.

### Anaerobic Processing and Storing of Microbiota

In alternative embodiments, microbiota used in compositions of the invention, or used to practice methods of the invention, are isolated, stored and/or cultured under suitably oxygen free (or substantially oxygen free). For example, in one embodiment, a fresh stool is transported via a stool collection device having a suitably oxygen free (or substantially oxygen free) appropriate container, e.g., a disposable leak proof ziplock/sealing bag. In alternative embodiments, the container can be made oxygen free by e.g., incorporating into the container a built in or clipped-on oxygen-scavenging mechanism, e.g., oxygen scavenging pellets as described e.g., in U.S. Pat. No. 7,541,091. In another embodiment, the container itself is made of an oxygen scavenging material, e.g., oxygen scavenging iron, e.g., as described by O2BLOCK™, or equivalents, which uses a purified and modified layered clay as a performance-enhancing carrier of oxygen-scavenging iron; the active iron is dispersed directly in the polymer. In one embodiment, oxygen-scavenging polymers are used to make the container itself or to coat the container, or as pellets to be added; e.g., as described in U.S. Pat. App. Pub. 20110045222, describing polymer blends having one or more unsaturated olefinic homopolymers or copolymers; one or more polyamide homopolymers or copolymers; one or more polyethylene terephthalate homopolymers or copolymers; that exhibit oxygen-scavenging activity. In one embodiment, oxygen-scavenging polymers are used to make the container itself or to coat the container, or as pellets to be added; e.g., as described in U.S. Pat. App. Pub. 20110008554, describing compositions comprising a polyester, a copolyester ether and an oxidation catalyst, wherein the copolyester ether comprises a polyether segment comprising poly(tetramethylene-co-alkylene ether). In one embodiment, oxygen-scavenging polymers are used to make the container itself or to coat the container, or as pellets to be added; e.g., as described in U.S. Pat. App. Pub. 201000255231, describing a dispersed iron/salt particle in a polymer matrix, and an oxygen scavenging film with oxygen scavenging particulates.

Alternatively, in addition to or in place of using an oxygen-scavenging mechanism, the air in the container is replaced (completely or substantially) with nitrogen and/or other inert non-reactive gas or gases. In alternative embodiments, the container simulates (creates) partially, substantially or completely an anaerobic environment.

In alternative embodiments, the stool (e.g., fecal sample) is held in an aesthetically acceptable container that will not leak nor smell yet maintain an anaerobic environment. In alternative embodiments, the container is sterile before receiving the fecal flora.

In alternative embodiments, a microbiota-containing container is maintained below room temperature, e.g., refrigerated, during most or all of its preparation, but not frozen; transportation and/or storage at e.g., a “stool bank” or at the site where the transplantation will take place. For example, once delivered to a “processing stool bank” it is stored in a cool room, cold container or refrigerator to minimize flora metabolism. In alternative embodiments, it is not frozen to prevent destruction of the bacterial cells of the microbiota-comprising formulation.

In alternative embodiments, stabilizing agents such as glycerol are added to the harvested and/or stored material. In one embodiment, the stool is frozen suddenly in liquid nitrogen or any similar coolant so e.g., it can be stored for prolonged periods of time while waiting processing.

In alternative embodiments, the stool is tested for various pathogens, as noted above. In alternative embodiments, once cleared of infective agents, it is homogenized and filtered to remove large particles of matter, then further processes, as described herein. In alternative embodiments, it is subdivided into desired volumes, e.g., which can be between 5 cc and 3 or more liters. For example, in one embodiment, a container comprises a 50 gram (g) stool, which can be held in an appropriate oxygen resistant plastic, e.g., a metallized polyethylene terephthalate polyester film, or a metallized MYLAR™.

In alternative embodiments, a composition of the invention is manufactured or processed under an inert gas cover or other anaerobic condition, and/or manufactured or processed in room air with some loss of activity. In alternative embodiments suitable gases include nitrogen, carbon dioxide, helium, neon, argon, krypton, xenon and/or radon.

### Defining or Prescreening Microbiota or Fecal Donors

In alternative embodiments, the methods of the invention comprise a step or prerequisite of pre-screening, or defining, the fecal, microbiota or FMT donor, e.g., using defined donors, as appropriate or required. In alternative embodiments, this is of advantage, especially in those types of products to be used e.g. in obesity, metabolic syndrome or diabetes, in addition to the screening out of individuals with extant infections. In one embodiment, the donor should ideally not have had antibiotics in childhood, as antibiotics in childhood are associated with obesity later in life because antibiotics alters the microbiota which no longer extract as much energy and also has other characteristics. In one embodiment, the donors are lean individuals. In alternative embodiments, the donor's age is between about 15 to 40 years of age, or about 10 to 50 years of age, or about 5 to 60 years of age.

In one embodiment, the defined donors measure naturally occurring high concentrations of Bacteroidetes and Firmicutes; some can also contain higher levels of Bacillus thuringienesis, e.g., B. thuringensis strain 4631 or a similar strain with bacterial activity against a C. difficile infection. Such donors could therefore contain thuricin CD, which in alternative embodiments is “added back”, e.g., to lower-concentration extracts if they did not contain it. In one embodiment, thuricin CD comprises a Trn-alpha or a Trn-beta. In one embodiment, wild type (WT) strains of B. thuringiensis are acceptable. In one embodiment, defined donors, especially in CDI treatment, would avoid stool from relatives as they may carry silent C. difficile infection. Furthermore, they would avoid people who have detectable methane on their breath i.e. methane producers, as methane production is generally associated with constipation-inducing bacteria.

### Packaging

The invention provides compositions, including preparations, formulations and/or kits, comprising combinations of ingredients, as described herein, for example, a frozen or freeze-dried liquid preparation or formulation of the invention and additional ingredients; or, a frozen or freeze-dried liquid preparation or formulation of the invention and a purified or substantially complete representation of a human microbiota. In alternative embodiments, these combinations can be mixed and administered together, or alternatively, they can be an individual member of a packaged combination of ingredients, e.g., as manufactured in a separate package, kit or container; or, where all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the pharmaceuticals of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one embodiment, the invention also provides a method of packaging where the compositions comprising combinations of ingredients of the invention are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item, (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two or three or more components (e.g., is a multi-ingredient combination of the invention): a thermoformed “blister” which houses multi-ingredient combination of the invention, and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (e.g., using an AERGO 8 DUO™, SCA Consumer Packaging, Inc., DeKalb Ill.) using regular heat seal tooling. This alternative aspect, using heat seal tooling, can seal common types of thermoformed packaging.

Blister Packaging

In alternative embodiments, combinations of ingredients of compositions of the invention, or combinations of ingredients for practicing methods of the invention, can be packaged alone or in combinations, e.g., as “blister packages” or as a plurality of packettes, including as lidded blister packages, lidded blister or blister card or packets or packettes, or a shrink wrap.

In alternative embodiments, laminated aluminium foil blister packs are used, e.g., for the preparation of drugs designed to dissolve immediately in the mouth of a patient. This exemplary process comprises having the drug combinations of the invention prepared as an aqueous solution(s) which are dispensed (e.g., by measured dose) into an aluminium (e.g., alufoil) laminated tray portion of a blister pack. This tray is then freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, e.g., using hard temper aluminium (e.g., alufoil) lidding material. In one aspect, hermetically-sealed high barrier aluminium (e.g., alufoil) laminates are used. In one aspect, any of the invention's products of manufacture, including kits or blister packs, use foil laminations and strip packs, stick packs, sachets and pouches, peelable and non-peelable laminations combining foil, paper, and film for high barrier packaging.

In alternative embodiments, any of the invention's multi-ingredient combinations or products of manufacture, including kits or blister packs, include memory aids to help remind patients when and how to take the drug. This safeguards the drug's efficacy by protecting each tablet, geltab or pill until it's taken; gives the product or kit portability, makes it easy to take a dose anytime or anywhere.

The invention will be further described with reference to the following examples; however, it is to be understood that the invention is not limited to such examples.

### EXAMPLES

### Example 1

### Exemplary “Rough Filtered” Compositions of the Invention

In one embodiment, an exemplary composition of the invention is largely (e.g., substantially) whole donor fecal material (e.g., stool) homogenized with saline as an extract of a human faeces. The biological material, e.g., donor fecal material (e.g., stool), is taken, dissolved and homogenised and passed through a sieve starting with a hole size of 2.0 mm, and then progressively passed through: 1.0 mm, 0.5 mm and finally down to 0.1 mm sieve holes. By stopping at to 0.1 mm sieve holes, this exemplary embodiment is in contrast to e.g., Sadowsky, et al., WO 2012/122478 A1, who prepared FMT material by filtering continued through ever smaller sieve holes until the stool was passed through a sieve down to 0.020 mm; this resulted in a very highly purified microbiota mass with well over 95% of bacterial cells alone, while the surrounding liquid material was discarded (the aim in bacterial cells alone formulations was to have essentially a bacteria-only composition, as it was recognised that CDI was largely cured by supplying Bacteroidetes and Firmicutes, and was not dependent in supplying any liquid components such as BAM's, see e.g., Khoruts A et al., J Clin Gastroenterol 44(5): 354-360 (2010). This produces a flora which is, at least for some applications, not optimal or defective because it is not physiological (i.e., lacks the native “liquid component”). This exemplary embodiment comprises use of a “rough filtered” composition to maintain a physiological status, and also, significantly, keeps the liquid components and small fibre, molecules to supply nutrients to the flora of the microbiota. In contrast to e.g., Sadowsky, et al., WO 2012/122478 A1 (using only the bacterial cells for carrying out such transplantation), in this exemplary embodiment the donor flora is left “incompletely” filtered (e.g., finally down to about 0.1 mm sieve holes) to allow for some physiological “food” to remain for the bacteria and to retain the liquid components with their anti-inflammatory products.

In alternative embodiments, this “incomplete filtering”, or “rough filtered” process and resultant product thereof also makes an FMT product of this invention much cheaper and/or easier, e.g., such that a patient can do this in their own home for self-administration.

In alternative embodiments, one or more cryoprotectants are added to this exemplary formulation of the invention, so that e.g., the extract can be frozen, and/or to produce a cheap format for home infusions by patients, e.g. with UC. Alternative exemplary features include preparation under cover of inert gases, and/or use of various “add in” or additions, as described above, including e.g., additions of prebiotics, probiotics and pre-treatment methods with antibiotics and biofilm-dissolving agents.

### Example 2

### Exemplary “High Level Filtration” Compositions of the Invention

In one embodiment, an exemplary composition of the invention comprises starting material from a donor from a defined donor pool (see below), where this donor contributes a stool that is centrifuges, then filtered with very high-level filtration using e.g., either metal sieving or Millipore filters, or equivalent, to ultimately permit only cells of bacterial origin to remain, e.g., often less than about 5 micrometres diameter. After the initial centrifugation, the solid material is separated from the liquid, and the solid is then filtered in progressively reducing size filters and tangential filters, e.g., using a Millipore filtration, and optionally, also comprising use of nano-membrane filtering. The filtering can also be done by sieves as described in WO 2012 122478, but in contrast using sieves that are smaller than 0.0120 mm, down to about 0.0110 mm, which ultimately result in having only bacterial cells present.

The supernatant separated during centrifugation is now taken and filtered progressively in a filtering, e.g., a Millipore filtering or equivalent systems, to end up with liquid which is finely filtered through an about 0.22 micron filter. This removes all particulate matter including all living matter, including bacteria and viruses. The product then is sterile, but the aim is to remove the bacteria but to keep their secretions, especially antimicrobial bacteriocins, bacteria-derived cytokine-like products and all accompanying Biologically Active Molecules (BAMs), including: thuricin (which is secreted by bacilli in donor stools), bacteriocins (including colicin, troudulixine or putaindicine, or microcin or subtilosin A), lanbiotics (including nisin, subtilin, epidermin, mutacin, mersacidin, actagardine, cinnamycin), lacticins and other antimicrobial or anti-inflammatory compounds.

In alternative embodiments, agents such as thuricin (which is secreted by bacilli in donor stools), nisin, lacticin and other BAMs (discussed above) are therefore extracted from the liquid portion of the donor stool and are preserved for ‘adding back’ to the cellular component. In alternative embodiments, synthetic or altered versions of these compositions. are “added back”.

In alternative embodiments, the supernatant extract “added back” also contains various peptides, micronutrients, protein, some fats, small carbohydrates, trace elements, mineral salts, ash, mucous, amino acids and other active agents, nutrients, vitamins or minerals, which can be added back to truly reconstitute a “wild type” healthy flora.

In alternative embodiments, this “supernatant extract” component, or synthetic equivalent thereof, is stored and/or pooled and used alone (without the bacterial cells), e.g., as a therapeutic, e.g., as an anti-inflammatory and/or anti-microbial agent.

In alternative embodiments, the invention recognizes the advantage and utility for using (as the “supernatant extract” component, or synthetic equivalent thereof) the various active molecules that the bacterial cells in stool produce which are able to not only kill Clostridia and other pathogens and their spores, but also heal UC and have other positive effects on conditions treated with this exemplary composition.

In alternative embodiments, a composition of the invention comprises extracted cells combined with their purified products, and/or a “supernatant extract” component, or synthetic equivalent thereof, which is then reconstituted for either freezing or freeze drying into a powder, or equivalent, before delivery to the patient.

In alternative embodiments, to achieve or preserve viability, these various added components are required or benefit from including a cryoprotectant, a lyoprotectant, or a preservative, e.g., as described in Example 3, below.

In alternative embodiments, at times this FMT product may also require topping up with components that may be required for a particular condition, disease or infection, e.g. adding more Firmicutes, Bacteroidetes and/or Bacillus (e.g., Bacillus thurigiensis) or others. The bacterial species can be isolated or separated by celltrifugation or plasmapharesis. In alternative embodiments, an exemplary composition of extracted complete (or substantially complete) human flora (a microbiota) is freeze-dried; and can also be formulated into a powder with various downstream applications.

In alternative embodiments, compositions of the invention are sieved or extracted total flora without the “crud” or non-functioning components, but for the first time also combining the active ingredients that have previously been removed by filtering, sieving and discarding. In alternative embodiments, compositions of the invention comprise the anti-bacterial agents and/or biologically active molecules produced by the microbiota organisms or found in the microbiota extract, e.g., which can act e.g., as interleukins, cytokines and the like, which are required or helpful in treatment of inflammation, especially ulcerative colitis.

### Example 3

### Exemplary “Cultured or Incubated” Compositions of the Invention

In one embodiment, an exemplary composition of the invention comprises cultured or incubated flora with the starting composition described in Examples 1 or 2. In one embodiment, a whole flora representative extract as in 1 or 2 (e.g., a substantially complete representation of a human microbiota) is prepared, e.g., as described herein, e.g., in Example 1 or Example 2, but then to incubate the FMT product for a variable time, e.g., about 2 to about 72 hours (hrs), or about 1 hour to 24 hours, or about 30 minutes to 12 hours, to increase the numbers of the bacteria and their products without needing to use larger numbers of donors. In alternative embodiments, the flora extract is incubated in a liquid enrichment culture medium in anaerobic conditions using appropriate nutrient broths of standard composition. These can then be aliquotted and frozen or freeze-dried (or lyophilized or cryodesiccated), thus increasing manufacturing volume of the FMT product rather than having to increase the volume of stool to be filtered from increasing number of donors. This would allow to produce a higher volume of very useful transplantation product.

A number of embodiments of the invention have been described. Nevertheless, it will be understood that various modifications may be made without departing from the spirit and scope of the invention. Accordingly, other embodiments are within the scope of the following claims.

1. A formulation or pharmaceutical preparation comprising:
(i) a liquid preparation harvested from a fecal material, wherein the liquid preparation is capable of passing through an at least about 0.22 micron filter and lacks any, or substantially all, intact viruses, fungal spores and bacteria, but retain bacteriophages; or
(ii) a liquid preparation made by a process comprising: (1) providing a fecal material; and (2) passing the fecal material through an at least about 0.22 micron (μ) filter such that the filtrate lacks any, or substantially all, intact viruses, fungal spores and bacteria, yet retain bacteriophages.

2. A formulation or pharmaceutical preparation comprising:
(a) (i) a highly filtered or substantially purified microbiota, and, (ii) a liquid preparation or formulation as set forth in claim 1;
(b) the formulation or pharmaceutical preparation of (a), wherein the highly filtered or substantially purified microbiota comprises or consists of a substantially isolated or a purified fecal flora or entire (or substantially entire) microbiota;
(c) the formulation or pharmaceutical preparation of (a) or (b), wherein the highly filtered or substantially purified microbiota comprises or consists of an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material;
(d) the formulation or pharmaceutical preparation of any of (a) to (c), wherein the highly filtered or substantially purified microbiota comprises or consists of a substantially isolated, purified, or substantially entire microbiota as described in Sadowsky, et al., WO 2012/122478 A1, or as described in Borody, et al., WO 2012/016287 A2;
(e) the formulation or pharmaceutical preparation of any of (a) to (d), wherein the highly filtered or substantially purified microbiota is made by using a plasmapheresis, a centrifugation, a celltrifuge, a column chromatography, an affinity chromatography, an immunoprecipitation, or antibodies fixed to a solid surface, a bead or a plate;
(f) the formulation or pharmaceutical preparation of any of (a) to (d), wherein the liquid preparation or formulation as set forth in claim 1 makes up between about 1% to 99%, or about 1%, 10%, 20%, 30%, 40%, 50%, 55%, 60%, 65%, 70%, 75%, 80%, 85%, 90% or 95% or more of the volume of a final formulation or pharmaceutical preparation;
(g) the liquid preparation of any of (a) to (f), further processed or formulated for either freezing or freeze-drying into a powder, or equivalent, and optionally further comprising a cryoprotectant, a lyoprotectant, or a preservative; or
(h) the liquid preparation of (g), further processed or formulated by reconstituting the frozen or freeze-dried powder in a liquid, wherein optionally the liquid is a sterile saline.

3. A formulation or pharmaceutical preparation comprising:
(a) (i) a “rough-”, “incomplete-” or medium-filtered microbiota-comprising fecal sample or isolate comprising or retaining its native or wild type physiological components or nutritive agents for bacteria of the microbiota,
wherein optionally the sample or isolate can pass through an about 0.1 mm sieve opening or filter hole; or
(ii) a “rough-”, “incomplete-” or medium-filtered microbiota-comprising fecal sample or isolate made by a process comprising:
(1) providing a fecal material; and
(2) passing the fecal material an about 0.1 mm sieve opening or filter hole;
(b) the “rough-”, “incomplete-” or medium-filtered microbiota-comprising fecal sample or isolate of (a), wherein the fecal sample or isolate consists of a human fecal material;
(c) the “rough-”, “incomplete-” or medium-filtered microbiota-comprising fecal sample or isolate of (a) or (b), further comprising by having added: a fiber, biologically active proteins or peptides, micronutrients, fats, sugars or small carbohydrates, trace elements, mineral salts, ash, mucous, amino acids, nutrients, vitamins or minerals, or all or any combination thereof, optionally “added back” to reconstitute a “wild type” healthy flora or human microbiota environment;
(d) the liquid preparation of any of (a) to (c), further processed or formulated for either freezing or freeze-drying into a powder, or equivalent, and optionally further comprising a cryoprotectant, a lyoprotectant, or a preservative; or
(f) the liquid preparation of (d), further processed or formulated by reconstituting the frozen or freeze-dried powder in a liquid, wherein optionally the liquid is a sterile saline.

4. A formulation or pharmaceutical preparation comprising:
(a) the formulation or pharmaceutical preparation of claim 1, wherein the bacteria or microbiota component has been cultured (or placed into an enrichment culture), or cultured (or placed into an enrichment culture) under anaerobic conditions, or harvested, stored and/or cultured (or placed into an enrichment culture) under anaerobic conditions,
wherein the highly filtered or substantially purified microbiota is cultured or placed into an enrichment culture before and after addition of the liquid preparation or formulation of claim 1;
(b) the formulation or pharmaceutical preparation of (a), wherein the bacteria or microbiota component is cultured for about 2 to about 72 hours (hrs), or about 1 hour to 24 hours, or about 30 minutes to 12 hours, to increase the numbers of the bacteria and their products without needing to use larger numbers of donors;
(c) the formulation or pharmaceutical preparation of (a) or (b), wherein the bacteria or microbiota component is cultured or incubated in a liquid enrichment culture medium in aerobic or in anaerobic conditions using appropriate nutrient broths; or
(d) the formulation or pharmaceutical preparation of any of (a) to (c), wherein the cultured bacteria or microbiota component is aliquotted and frozen or freeze-dried or lyophilized or cryodesiccated.

5. The formulation or pharmaceutical preparation of claim 1, further comprising, or having added to: at least one bacteria or species of a Firmicutes, Bacteroidetes, a Bacillus, or a Bacillus thurigiensis, wherein optionally the Firmicutes, Bacteroidetes, a Bacillus is from a culture.

6. The formulation or pharmaceutical preparation of claim 1, further comprising, or having added to: at least one probiotic or prebiotic, wherein optionally the prebiotic comprises an inulin, lactulose, extracts of artichoke, chicory root, oats, barley, various legumes, garlic, kale, beans or flacks or an herb,
wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E. coli, a Strep fecalis and equivalents.

7. The formulation or pharmaceutical preparation of claim 1, further comprising, or having added to: at least one congealing agents, wherein optionally the congealing agent comprises an arrowroot or a plant starch, a powdered flour, a powdered potato or potato starch, an absorbant polymer, an Absorbable Modified Polymer (AMP®), EndoClot, Santa Clara, Calif.), and/or a corn flour or a corn starch.

8. The formulation or pharmaceutical preparation of claim 1, further comprising, or having added to: at least one an anti-inflammatory agent, wherein optionally the inflammatory agent comprises or is a 4 or a S-amino-salicylate, an olsalazine (e.g., DIPENTUM™), a mesalazine (also known as mesalamine or a 5-aminosalicylic acid (5-ASA), e.g., ASACOL™ or LIALDA™), a sulfasalazine (e.g., AZULFIDINE™, SALAZOPYRIN™ or SULAZINE™), and/or a balsalazide (e.g. COLAZAL™ or COLAZIDE™), or an equivalent thereof or a combination thereof.

9. The formulation or pharmaceutical preparation of claim 1, further comprising, or having added to: at least one opiate inhibitor or opiate antagonist, wherein optionally the opiate inhibitor or opiate antagonist is a methylnaltrexone bromide, a naltrexone (e.g., REVIA™, DEPADE™, VIVITROL™), or a nalmefene glucuronide.

10. The formulation or pharmaceutical preparation of claim 1, further comprising, or having added to: at least one acid suppressant, antacid and/or proton pump inhibitor, wherein optionally the acid suppressant is an H2 Receptor Antagonist, wherein optionally the H2 Receptor Antagonist is a cimetidine (e.g., TAGAMET™), a ranitidine (e.g., ZANTAC™), or an equivalent, wherein optionally the Proton Pump Inhibitor is an omeprazole (e.g., LOSEC™, ANTRA™, GASTROLOC™, MOPRAL™, OMEPRAL™, PRILOSEC™), an esameprazole (e.g., NEXIUM™), a pantoprazole (e.g., SOMAC™, TECTA™, PANTOLOC™, PROTIUM™, PROTONIX™) and equivalents.

11. The formulation or pharmaceutical preparation of claim 1, further comprising an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.

12. The formulation or pharmaceutical preparation of claim 1, further comprising, or having added to: at least one Biofilm Disrupting Compound, wherein optionally the biofilm disrupting compound comprises an enzyme, a deoxyribonuclease (DNase), N-acetylcysteine, an alginate lyase, glycoside hydrolase dispersin B; Quorum-sensing inhibitors e.g., ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.

13. The formulation or pharmaceutical preparation of claim 1, wherein the formulation or pharmaceutical preparation is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, such as EUDRAGIT S™ (Evonik Industries AG, Essen, Germany), which dissolves at pH 7 or greater, e.g., comprises a multimatrix (MMX) formulation.

14. A delivery vehicle, product of manufacture, container, syringe, device or bag, comprising: a formulation or pharmaceutical preparation of claim 1.

15. A delivery vehicle, formulation, composition, pharmaceutical preparation, product of manufacture, container, bag or device comprising: a formulation or pharmaceutical preparation of claim 1, initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

16. A method for the amelioration, stabilization, treatment and/or prevention of an infection, disease, treatment, poisoning or a condition having a bowel dysfunction component or side-effect comprising administering to an individual in need thereof via a delivery vehicle, formulation, composition, pharmaceutical preparation, product of manufacture, container or device comprising: a formulation or pharmaceutical preparation of claim 1.

17. The method of claim 16, wherein the infection, disease, treatment, poisoning or condition having a bowel dysfunction component or side-effect comprises a constipation, an inflammatory bowel disease (IBD), Crohn's disease, hepatic encephalopathy, enteritis, colitis, irritable bowel syndrome (IBS), fibromyalgia (FM), chronic fatigue syndrome (CFS), depression, attention deficit hyperactivity disorder (ADHD), multiple sclerosis (MS), systemic lupus erythematosus (SLE), travelers' diarrhea, small intestinal bacterial overgrowth, chronic pancreatitis, a pancreatic insufficiency, exposure to a poison or a toxin or for an infection, a toxin-mediated traveler's diarrhea, a poisoning, a pseudomembranous colitis, a Clostridium infection, a C. perfringens welchii or a Clostridium difficile infection, a neurological condition, Parkinson's disease, myoclonus dystonia, autism, amyotrophic lateral sclerosis or multiple sclerosis, Grand mal seizures or petit mal seizures, a halitosis, a hepato-renal syndrome and/or a diverticulitis or a recurrent diverticulitis, an atopic condition, an asthma, an Attention Deficit Disorder (ADD and ADHD), an obsessive compulsive disorder (OCD), a depression, a schizophrenia and/or a mood disorder.

18. A method for the amelioration, stabilization, treatment and/or prevention of, or decreasing or delaying the symptoms of, an infection, disease, treatment, poisoning or a condition having a bowel dysfunction component or side-effect, or for the amelioration, treatment and/or prevention of a constipation, for the treatment of an abdominal pain, a non-specific abdominal pain or a diarrhea, a diarrhea caused by: a drug side effect or a psychological condition or Crohn's Disease, a poison, a toxin or an infection, a toxin-mediated traveller's diarrhea, or a Clostridium or a C. perfringens welchii or a C. difficile infection or a pseudo-membranous colitis associated with a Clostridium infection, or for preventing, or decreasing or delaying the symptoms of, or ameliorating or treating individuals with spondyloarthropathy, spondylarthritis or sacrolileitis (an inflammation of one or both sacroiliac joints); a nephritis syndrome; an inflammatory or an autoimmune condition having a gut or an intestinal component; lupus; irritable bowel syndrome (IBS or spastic colon); or a colitis; Ulcerative Colitis or Crohn's Colitis; constipation; autism; a degenerative neurological diseases; amyotrophic lateral sclerosis (ALS), Multiple Sclerosis (MS) or Parkinson's Disease (PD); a Myoclonus Dystonia; Steinert's disease; proximal myotonic myopathy; an autoimmune disease; Rheumatoid Arthritis (RA) or juvenile idiopathic arthritis (HA); Chronic Fatigue Syndrome; benign myalgic encephalomyelitis; chronic fatigue immune dysfunction syndrome; chronic infectious mononucleosis; epidemic myalgic encephalomyelitis; obesity; hypoglycemia, pre-diabetic syndrome, type I diabetes or type II diabetes; Idiopathic thrombocytopenic purpura (ITP); an acute or chronic allergic reaction; hives, a rash, a urticaria or a chronic urticaria; and/or insomnia or chronic insomnia, Grand mal seizures or petit mal seizures, a halitosis, a hepato-renal syndrome and/or a diverticulitis or a recurrent diverticulitis, an atopic condition, an asthma, an Attention Deficit Disorder (ADD and ADHD), an obsessive compulsive disorder (OCD), a depression, a schizophrenia and/or a mood disorder, comprising:
administering to an individual in need thereof via a formulation or pharmaceutical preparation of claim 1, in single, repeat or multiple administrations, deliveries or infusions.

19-20. (canceled)

21. A formulation or pharmaceutical preparation comprising: (a) the formulation or pharmaceutical preparation of claim 2,
wherein the bacteria or microbiota component has been cultured (or placed into an enrichment culture), or cultured (or placed into an enrichment culture) under anaerobic conditions, or harvested, stored and/or cultured (or placed into an enrichment culture) under anaerobic conditions,
wherein optionally the highly filtered or substantially purified microbiota and liquid preparation or formulation of claim 2 is cultured or placed into an enrichment culture;
(b) the formulation or pharmaceutical preparation of (a), wherein the bacteria or microbiota component is cultured for about 2 to about 72 hours (hrs), or about 1 hour to 24 hours, or about 30 minutes to 12 hours, to increase the numbers of the bacteria and their products without needing to use larger numbers of donors;
(c) the formulation or pharmaceutical preparation of (a) or (b), wherein the bacteria or microbiota component is cultured or incubated in a liquid enrichment culture medium in aerobic or in anaerobic conditions using appropriate nutrient broths; or
(d) the formulation or pharmaceutical preparation of any of (a) to (c), wherein the cultured bacteria or microbiota component is aliquotted and frozen or freeze-dried or lyophilized or cryodesiccated.

22. The formulation or pharmaceutical preparation of claim 1, wherein:
(a) in step (i), the fecal material is passed through a series of progressively smaller sized filters before the resulting liquid preparation is finally passed through the at least about 0.22 micron filter,
(b) before passing the fecal material through the at least about 0.22 micron filter, the fecal material is first centrifuged, and the supernatant is used as the liquid preparation starting material for step (i) or (ii),
(c) before passing the fecal material through an at least about 0.22 micron filter, and/or before centrifuging, the fecal material is first homogenized with a saline or a buffered solution,
(d) before passing the fecal material through an at least about 0.22 micron filter, the starting fecal material, or the after-centrifugation supernatant, is filtered with one or several filters to ultimately remove all (or substantially all) cells of bacterial origin from the liquid preparation, or to ultimately remove all cells (or substantially all) of less than about 5 micrometres (μπ [symbol] ) diameter from the liquid preparation,
(e) in the liquid preparation the fecal material consists of a human fecal material;
(f) the liquid preparation further comprising by having added to the liquid preparation: a fiber, biologically active proteins or peptides, micronutrients, fats, sugars or small carbohydrates, trace elements, mineral salts, ash, mucous, amino acids, nutrients, vitamins or minerals, or all or any combination thereof, optionally “added back” to reconstitute a “wild type” healthy flora or human microbiota environment;
(g) the liquid preparation is further processed or formulated for either freezing or freeze-drying into a powder, or equivalent, and optionally further comprising a cryoprotectant, a lyoprotectant, or a preservative; or
(h) the liquid preparation is further processed or formulated by reconstituting the frozen or freeze-dried powder in a liquid, wherein optionally the liquid is a sterile saline.
